(19) United States
(12) Patent Application Publication
(10) Pub. No.: US 2026/0166078 A1
(43) Pub. Date: Jun. 18, 2026

(54) Preparing method for artificial blood

(71) Applicant: Yikang Li, Foshan (CN)

(72) Inventor: Jinhuan Tan, Qingyuan (CN)

(21) Appl. No.: 18/979,892

(22) Filed: Dec. 13, 2024

(51) Int. Cl. A61K 35/14

(52) U.S. Cl. CPC A61K 35/14

(57) ABSTRACT

A preparing method for artificial blood includes the following steps: (1) add raw materials into a tank reactor, mix with solvent to obtain a mixture; (2) add glycerol, lecithin, soybean oil, ethyl cellulose, TritonX-100, and Tween-40 in a high shear milk machine; control the high shear milk machine to react for 30 min, then stir to obtain a white emulsion suspension; (3) take 100 g of the mixture, add 1000 ml of physiological saline and mix well as a diluent; dilute the white emulsion suspension, then heat to 90° C. and cool to 10° C. to form an amino acid solution; (4) inject oxygen into the amino acid solution for dissolved oxygen treatment for 25 minutes; (5) measuring the amino acid solution, and when the partial pressure of oxygen reaches 105 Kpa or more, obtain the artificial blood.

### FIELD OF THE INVENTION

The present invention relates to the technical field of artificial blood, particularly to a preparation method of artificial blood.

### BACKGROUND OF THE INVENTION

World Health Organisation Statistics: there are 11,144 million units of donated blood worldwide each year, but this quantity is far from being insufficient for clinical use. With the increasing requirement of social blood and the increasing conflict that the donated blood quantity is far from meeting the requirement, a replacement for human blood has been constantly sought from the beginning of the last century in the world. It is expected that blood stasis can be solved by artificial blood and blood transfusion infection diseases can be avoided.

In 2013, a Romanian scientist developed an artificial blood—a material synthesized from water, inorganic salt, and centipede hemoglobin extracted from deep-sea insects in vivo, which can replace blood for a short time to achieve oxygen and carbon dioxide exchange metabolism. As in December, Japanese researchers have succeeded in using stem cells to grow red blood cells capable of carrying oxygen. On this basis, red blood cells used for blood transfusion can be heavily cultivated, thereby helping medical systems relieve blood stasis.

In 1980, scientific workers at the Research Institute of Shanghai Organic Chemistry in the Chinese Academy of Sciences and the Third Military Medical University of the Chinese People's Liberation Army successfully developed artificial blood after 5 years of research. The artificial blood is a superfine emulsion of fluorocarbon in water. After the superfine emulsion of fluorocarbon is injected into a human body, like red blood cells in normal blood of the human body, it has good oxygen-carrying capacity and carbon dioxide discharging capacity. It can be said that the superfine emulsion of fluorocarbon is a substitute for red blood cells. Fluorocarbon compounds, like Crab's claws, can grasp the oxygen, and then release the oxygen in the human body, so as to perform a special redox reaction in the human body. The biochemical property thereof is very stable, and regardless of the blood type, artificial blood can be used.

In the prior art, artificial blood mainly replaces a human blood part function, such as hemoglobin or hemoglobin and platelets, so as to play a role of temporarily replacing part of blood. The artificial blood is mainly used for emergency blood use by a trauma operator. The artificial blood cannot improve the hematopoietic capacity of the patient, and cannot completely replace blood. The additive thereof needs to be discharged outside the body for several weeks. There is no direct injection or oral administration in the prior art for promoting the production of more hematopoietic stem cells from the bone marrow of a human body, so as to differentiate to obtain, for example, red blood cells, white blood cells, platelets, and the like, thereby improving the autopoietic function.

### SUMMARY OF THE INVENTION

To overcome the deficiencies of the prior art, the present invention provides a preparation method for artificial blood. The main components of the prepared artificial blood are composed of essential amino acids, trace elements, vitamins, nutrients, and growth factors, which can improve the hematopoietic capacity of a patient through intestinal absorption.

The technical solution of the present invention is as follows: a preparing method for artificial blood, per 100 g of artificial blood including raw materials as follows: 12-15 g of essential amino acids, 6.5-7.0 g of trace elements, 23-23.5 g of nutrients, and 0.05-0.06 mg of vitamins; comprising the following steps:
(1) add the raw materials into a tank reactor, and mix with solvent to obtain a mixture;
(2) add glycerol, lecithin, soybean oil, ethyl cellulose, TritonX-100, and Tween-40 in a high shear milk machine in a mass ratio of 2:2:2:1:0.05:0.05; control the high shear milk machine to react at 20° C. for 30 min, then stir with microwave for 40 seconds to obtain a white emulsion suspension;
(3) take 100 g of the mixture obtained in step (1), add 1000 ml of physiological saline, and mix well as a diluent; dilute the white emulsion suspension obtained in step (2) with the diluent at a ratio of 1:25, then heat to 90° C. and cool to 10° C. under vacuum to form an amino acid solution wrapped in emulsion particles;
(4) inject a mixture of medical sterile pure O₂ and O₃ at 2-3 atmospheres of pressure or oxygen undergoing photochemical treatment using a medical high oxygen liquid therapy device at a flow rate of 4-5.5 L/min into the amino acid solution obtained in step (3) for dissolved oxygen treatment for 25 minutes;
(5) measuring the amino acid solution after the dissolved oxygen treatment obtained in step (4) using a blood gas analyzer, and when the partial pressure of oxygen reaches 105 Kpa or more, filling same into an infusion bag at equal pressure to obtain the artificial blood.

In an embodiment, in 100 g of artificial blood, the essential amino acids include arginine 0.0058 g lysine 0.0026 g, tyrosine<0.0038 g, leucine 0.0032 g, aspartic acid 0.11 g, valine 0.0036 g, isoleucine 0.0018 g.

In 100 g of artificial blood, the trace elements include vanadium 0.00260 mg, manganese 0.0706 mg, iron 6.60 mg, cobalt 0.00106 mg, nickel less than 0.05 mg, copper less than 0.02 mg, lithium less than 0.02 mg, arsenic 0.00724 mg, selenium less than 0.003 mg, rubidium 0.0434 mg, molybdenum less than 0.003 mg.

In 100 g of artificial blood, the nutrients include total flavone 7.88 mg, folate<0.0015 g, triterpenes<2.61 g on an oleanolic acid basis, fructooligosaccharides<0.103 mg, beta carotene<1.5 μg, fructose 3.4 g, glucose 13.4 g, sucrose 3.7 g, niacin<100 μg.

In 100 g of artificial blood, the vitamins include L (+) ascorbic acid<0.4 mg, vitamin A<0.35 mg, vitamin B12<0.05 mg.

In 100 g of artificial blood, the growth factors include human stem cell growth factor SCF1.5×10⁻⁵ mg, recombinant human erythropoietin EPO1.2×10⁻⁵ mg, recombinant human platelet growth factor TPO1.2×10⁻⁵ mg, recombinant human interleukin IL-11 112.5×10⁻⁵ mg.

In an embodiment, in 100 g of artificial blood, the essential amino acids include methionine<0.0030 g, glycine<0.0048 g, glutamic acid<0.018 g, leucine<0.0032 g, serine<0.0055 g, valine<0.0036 g, isoleucine<0.0018 g, histidine<0.0018 g;
the trace elements include vanadium 0.00260 mg, manganese 0.0706 mg, iron 6.60 mg, cobalt 0.00106 mg, nickel less than 0.05 mg, copper less than 0.02 mg, lithium less than 0.02 mg, arsenic 0.00724 mg, selenium less than 0.003 mg, rubidium 0.0434 mg, molybdenum less than 0.003 mg;
the nutrients included total flavone 7.88 mg, folate<0.0015 g, triterpenoids<2.61 g, fructooligosaccharides 0.103 mg/100 ml, beta carotene<1.5 μg, fructose 3.4 g, glucose 13.4 g, sucrose 3.7 g, Niacin<100 μg;
the vitamins include L (+) ascorbic acid<0.4 mg, vitamin A<0.35 mg, vitamin B12<0.05 mg;
the growth factors include human stem cell growth factor SCF1.5×10⁻⁵ mg, recombinant human erythropoietin EPO1.2×10⁻⁵ mg.

In an embodiment, in 100 g of artificial blood, the essential amino acids include phenylalanine 0.0044 g, alanine 0.0055 g, proline<0.12 g, leucine 0.0032 g, aspartic acid 0.11 g, valine 0.0036 g, isoleucine 0.0018 g;
the trace elements include vanadium 0.0260 mg, manganese 0.706 mg, iron 66.0 mg, cobalt 0.0106 mg, nickel<0.5 mg, copper<0.2 mg, lithium<0.2 mg, arsenic 0.0724 mg, selenium<0.03 mg, rubidium 0.434 mg, molybdenum<0.03 mg.
the vitamins includes L (+) ascorbic acid<0.4 mg, vitamin A<0.35 mg, vitamin B12<0.05 mg;
the growth factors comprise human stem cell growth factor SCF1.5×10⁻⁵ mg.

The trace elements come from organic compounds extracted from food or other forms of compounds that are easily absorbed by the human body.

The amount of iron added is 55 mg/100 g.

The amount of L (+) ascorbic acid added to the vitamin is 0.35 mg/100 g, and the amount of vitamin A added is 0.30 mg/100 g.

In a mixture of pure O₂ and O₃, the ratio of pure O₂ to O₃ is 3.54:1 by volume percentage.

The beneficial effects of the present invention are:

1. The artificial blood produced by the method for preparing artificial blood in the present invention has no fluorocarbon compounds, which are safe, reliable, and have no side effects.

2. The artificial blood produced by the method for preparing artificial blood in the present invention can be used for people of various blood types, and there will be no serious hemolysis reaction after transfusion. Especially in rescue situations, time is life, and it can be used immediately without checking the blood type or conducting cross-matching tests. For large-scale on-site first aid, it is also simple and fast.

3. The artificial blood produced by the preparing method of artificial blood in the present invention is easy to store and need not be stored in the refrigerator at 4-6° C. like the blood of a blood donor. The artificial blood does not contain components such as hemoglobin and platelets and is not easily spoiled or coagulated. In addition, the triterpenoid component can inhibit the toxic activity of diseases, and have anti-inflammatory, antibacterial effects, and anticoagulant effects. It can be stored for several months to several years.

5. The artificial blood produced by the method for preparing artificial blood in the present invention will not cause cross-infection. Usually, if blood transfusion is not thoroughly checked, some bacteria and viruses may be introduced into the recipient's body, causing cross-infection. However, the artificial blood is produced industrially and can not contain any bacteria or viruses.

6. After the donated blood is injected into the patient's body, it takes 24 hours to restore the ability to carry oxygen. The artificial blood produced by the preparing method of artificial blood described in the present invention can immediately achieve the function of transporting oxygen. It is formed by mixing glycerol, lecithin, soybean oil high shear emulsion, and diluent containing amino acids to form the amino acid solution wrapped in emulsion particles. After dilution, the emulsion particles in the solution have oxygen carrying capacity, which can further improve the ability to dissolve the mixed gas of O₂ and O₃. Finally, the oxygen partial pressure reaches above 105 Kpa, improving the oxygen carrying capacity of the artificial blood, which can reach the same oxygen carrying capacity as perfluoroalkane emulsion. O₃ can also be decomposed into oxygen atoms and oxygen in the blood, which can supplement oxygen and eliminate free radicals, and also has bactericidal and antiviral effects.

7. The artificial blood produced by the method for preparing artificial blood described in the present invention can supplement the necessary amino acids, trace elements, vitamins, nutrients, and growth factors for the proliferation process of hematopoietic stem cells. Among them, leucine, isoleucine, and valine are branched chain amino acids that cannot be synthesized by the human body and can only be supplemented through food. They are also essential for the synthesis of hematopoietic stem cells, and their content plays an important role in the metabolism and maintenance of dryness of hematopoietic stem cells. Proline, lysine, and other essential amino acids for protein synthesis play an important role in the synthesis of hematopoietic cells and hemoglobin.

The trace element iron in the artificial blood produced by the preparing method of artificial blood according to the present invention is a necessary raw material for synthesizing hemoglobin and also the structural center of hemoglobin in the hemoglobin, which is a key part responsible for binding with oxygen and plays an important role in the synthesis of red blood cells and oxygen carrying capacity after artificial blood enters the human body. On the other hand, iron can promote the synthesis of hemoglobin peptides and increase the amount of hemoglobin.

In addition, copper takes part in hematopoiesis and enzyme synthesis, while nickel promotes iron absorption and stimulates hematopoietic function; lithium can improve hematopoietic function; vanadium can stimulate the hematopoietic system and promote the formation of hemoglobin; cobalt can activate metabolism, promote hemoglobin synthesis, and increase red blood cells; manganese can promote hematopoiesis; arsenic can promote the hematopoietic function of bone marrow by stimulating hematopoietic cells through red blood cell products; selenium protects the hematopoietic system; rubidium is beneficial for improving the function of the hematopoietic system; molybdenum has the function of promoting hematopoiesis, metabolism, and ensuring smooth blood flow.

In nutrients, hematopoietic stem cells use vitamin C to regulate the abundance of certain chemical modifications on DNA (as part of the epigenome). Hematopoietic stem cells require very high levels of vitamin C, which can also promote iron absorption in the intestine. Vitamin A cannot be synthesized by the human body. Consuming a large amount of vitamin A in the diet can produce yellow acid, which can effectively activate hematopoietic cells. Lack of vitamin A leads to the loss of differentiation of hematopoietic stem cells. Cobalamine in vitamin B12 can promote the development and maturation of red blood cells, which drives the microbial synthesis of nucleotides and ensures normal hematopoietic function in the body. Niacin participates in hematopoiesis by promoting iron absorption; total flavonoids protect the hematopoietic system; folic acid can promote the synthesis of red blood cells and white blood cells, which is an important hematopoietic material, and can enhance immune function. β-carotene has hematopoietic function and can improve anemia and cold-blooded syndrome. It can further promote hematopoiesis by catalyzing the production of vitamins through iron.

Among growth factors, human stem cell growth factor SCF, recombinant human erythropoietin EPO, recombinant human platelet growth factor TPO, and recombinant human interleukin IL-11 together promote the generation of hematopoietic stem cells in the blood. They further stimulate hematopoietic stem cells to use raw materials for division and proliferation, thereby improving the ability of red blood cell division and platelet synthesis in the blood. Together with the essential amino acids, the trace elements, the vitamins, and the nutrients, they synergistically promote blood recovery ability.

### DETAILED DESCRIPTION OF ILLUSTRATED EMBODIMENTS

In order to clarify the invention purpose, technical solution, and technical effect of the present invention, further explanation of the present invention will be provided below in conjunction with specific embodiments. It should be understood that the specific embodiments described herein are only for explaining the present invention and are not intended to limit the present invention.

A preparing method for artificial blood, per 100 g of artificial blood including raw materials as follows: 12-15 g of essential amino acids, 6.5-7.0 g of trace elements, 23-23.5 g of nutrients, and 0.05-0.06 mg of vitamins; comprising the following steps:
(1) add the raw materials into a tank reactor, and mix with solvent to obtain a mixture;
(2) add glycerol, lecithin, soybean oil, ethyl cellulose, TritonX-100, and Tween-40 in a high shear milk machine in a mass ratio of 2:2:2:1:0.05:0.05; control the high shear milk machine to react at 20° C. for 30 min, then stir with microwave for 40 seconds to obtain a white emulsion suspension;
(3) take 100 g of the mixture obtained in step (1), add 1000 ml of physiological saline, and mix well as a diluent; dilute the white emulsion suspension obtained in step (2) with the diluent at a ratio of 1:25, then heat to 90° C. and cool to 10° C. under vacuum to form an amino acid solution wrapped in emulsion particles;
(4) inject a mixture of medical sterile pure O₂ and O₃ at 2-3 atmospheres of pressure or oxygen undergoing photochemical treatment using a medical high oxygen liquid therapy device at a flow rate of 4-5.5 L/min into the amino acid solution obtained in step (3) for dissolved oxygen treatment for 25 minutes;
(5) measuring the amino acid solution after the dissolved oxygen treatment obtained in step (4) using a blood gas analyzer, and when the partial pressure of oxygen reaches 105 Kpa or more, filling same into an infusion bag at equal pressure to obtain the artificial blood.

In an embodiment, in 100 g of artificial blood, the essential amino acids include arginine 0.0058 g lysine 0.0026 g, tyrosine<0.0038 g, leucine 0.0032 g, aspartic acid 0.11 g, valine 0.0036 g, isoleucine 0.0018 g;
the trace elements include vanadium 0.00260 mg, manganese 0.0706 mg, iron 6.60 mg, cobalt 0.00106 mg, nickel less than 0.05 mg, copper less than 0.02 mg, lithium less than 0.02 mg, arsenic 0.00724 mg, selenium less than 0.003 mg, rubidium 0.0434 mg, molybdenum less than 0.003 mg;
the nutrients include total flavone 7.88 mg, folate<0.0015 g, triterpenes<2.61 g on an oleanolic acid basis, fructooligosaccharides<0.103 mg, beta carotene<1.5 μg, fructose 3.4 g, glucose 13.4 g, sucrose 3.7 g, niacin<100 μg;
the vitamins includes L (+) ascorbic acid<0.4 mg, vitamin A<0.35 mg, vitamin B12<0.05 mg;
the growth factors comprise human stem cell growth factor SCF1.5×10⁻⁵ mg, recombinant human erythropoietin EPO1.2×10⁻⁵ mg, recombinant human platelet growth factor TPO1.2×10⁻⁵ mg, recombinant human interleukin IL11 12.5×10⁻⁵ mg.

In an embodiment, in 100 g of artificial blood, the essential amino acids include methionine<0.0030 g, glycine<0.0048 g, glutamic acid<0.018 g, leucine<0.0032 g, serine<0.0055 g, valine<0.0036 g, isoleucine<0.0018 g, histidine<0.0018 g;
the trace elements include: vanadium 0.00260 mg, manganese 0.0706 mg, iron 6.60 mg, cobalt 0.00106 mg, nickel less than 0.05 mg, copper less than 0.02 mg, lithium less than 0.02 mg, arsenic 0.00724 mg, selenium less than 0.003 mg, rubidium 0.0434 mg, molybdenum less than 0.003 mg;
the nutrients include total flavone 7.88 mg, folate<0.0015 g, triterpenoids<2.61 g, fructooligosaccharides 0.103 mg/100 ml, beta carotene<1.5 μg, fructose 3.4 g, glucose 13.4 g, sucrose 3.7 g, Niacin<100 μg;
the vitamins include L (+) ascorbic acid<0.4 mg, vitamin A<0.35 mg, vitamin B12<0.05 mg;
the growth factors comprise human stem cell growth factor SCF1.5×10⁻⁵ mg, recombinant human erythropoietin EPO1.2×10⁻⁵ mg.

In an embodiment, in 100 g of artificial blood, the essential amino acids include phenylalanine 0.0044 g, alanine 0.0055 g, proline<0.12 g, leucine 0.0032 g, aspartic acid 0.11 g, valine 0.0036 g, isoleucine 0.0018 g;
the trace elements include vanadium 0.0260 mg, manganese 0.706 mg, iron 66.0 mg, cobalt 0.0106 mg, nickel<0.5 mg, copper<0.2 mg, lithium<0.2 mg, arsenic 0.0724 mg, selenium<0.03 mg, rubidium 0.434 mg, molybdenum<0.03 mg;
the vitamins includes L (+) ascorbic acid<0.4 mg, vitamin A<0.35 mg, vitamin B12<0.05 mg;
the growth factors include human stem cell growth factor SCF1.5×10⁻⁵ mg.

The trace elements come from organic compounds extracted from food or other forms of compounds that are easily absorbed by the human body.

The amount of iron added is 55 mg/100 g.

The amount of L (+) ascorbic acid added to the vitamin is 0.35 mg/100 g, and the amount of vitamin A added is 0.30 mg/100 g.

In a mixture of pure O₂ and O₃, the ratio of pure O₂ to O₃ is 3.54:1 by volume percentage.

The demonstration experiment of the promoting effect of the artificial blood produced by the method for preparing artificial blood on hematopoietic stem cells is as follows:
I. Add CD34+ cells to human hematopoietic culture medium containing 1.5×10⁻⁵ mg/100 g SCF, 1.2×10⁻⁵ mg/100 g TPO, 1.2×10⁻⁵ mg/100 g EPO, and 2.5×10⁻⁵ mg/100 g IL-11, and inoculate them into a 24 well plate (2.0×10⁵/well) with a cell density of 1.0×10⁴/ml and a culture system of 1 ml per well. Add 5 μg/ml of the artificial blood to the experimental group for hematopoiesis. The control group was not added hematopoietic cells. Each group has three holes. The 24 well plate was incubated in a 37° C., 5% CO₂ incubator for 12 days. After 12 days, the cell count was taken, and the results are shown in Table 1:

TABLE 1
Cell proliferation statistics results

| | The Growth factor of | The Growth factor of |
|---|---|---|
| | Nucleated cell | CD34 + cells |
| The experimental group | 79.6 | 10.3 |
| The control group | 48.7 | 5.4 |

The experimental results showed that artificial blood can promote the proliferation of nucleated cells, and compared with the control group, human hematopoiesis can significantly increase the number of CD34+ cells.

### II Efficacy Experiment of the Artificial Blood on Hemorrhagic Anemia Mice

Establishment of hemorrhagic anemia mouse model: Bleed mice in small amounts multiple times to achieve anemia, thereby simulating clinical hemorrhagic anemia. Mice were randomly divided into 10 groups based on body weight, with 8 mice in each group, each mouse was given a normal diet, and the hemoglobin and red blood cell counts were below the lower limit of normal values, which were 11.8 g/dl, 7.3×10⁶/μl, and 41.2%, respectively, a hemorrhagic anemia animal model was established.

### 1. Experimental Methods

(1) After the experimental mice lost blood, they were immediately intragastric administration at a dose of 25 ml/kg body weight, once a day, for 5 consecutive days.

The artificial blood was given to the mice in each control group as follows:
control group I: excluding growth factors from the artificial blood;
control group II: excluding nutrients from the artificial blood;
control group III: excluding iron and growth factors from the artificial blood;
control group IV: excluding trace elements and growth factors from the artificial blood;
control group V: containing only essential amino acids in the artificial blood components;
control group VI: containing only trace elements in the artificial blood components; control group VII: containing only vitamins in the artificial blood components;
control group VIII: excluding TPO and IL-11 components of growth factors from the artificial blood;
control group IX: Administe physiological sali

Blood was collected from the tail vein of mice at Oh, 20 h, 42 h, 68 h, and 92 h, and hemoglobin and red blood cells were measured using a hemocytometer. Administer for 0 hours as a control before blood loss.

(2) After the experimental mice lost blood, they were immediately administered intravenously at a dose of 25 ml/kg body weight once a day for 5 consecutive days. Comparative Example 1 was given intravenous injection of perfluoronaphthalene emulsion, while comparative example 2 was given intravenous injection of physiological saline. After intravenous injection at 30 minutes, 60 minutes, and 100 minutes, respectively, observe the blood oxygen concentration.

### 2. Experimental Result: Refer to Table 2-4

TABLE 2
Effect of the artificial blood on haemoglobin
in haemolytic anemia mice
Hemoglobin (g/dl)

| | 0 h | 20 h | 42 h | 68 h | 92 h |
|---|---|---|---|---|---|
| the artificial | 17 | 13.5 | 14.9 | 17.2 | 20.5 |
| blood | | | | | |
| control group I | 17 | 11.2 | 13.5 | 15.3 | 19.3 |
| control group II | 17 | 12.2 | 14.7 | 16.3 | 19.9 |
| control group III | 17 | 10 | 11.6 | 12.5 | 18.1 |
| control group IV | 17.1 | 10.2 | 11.6 | 12.1 | 17.9 |
| control group V | 17 | 10.4 | 11.2 | 11.5 | 17.6 |
| control group VI | 17.1 | 9.8 | 10.5 | 11.5 | 17.5 |
| control group VII | 17.1 | 9.4 | 9.8 | 11.2 | 17.4 |
| control group VIII | 17 | 12.8 | 14.5 | 16.3 | 19.8 |
| control group X | 17 | 9 | 9.6 | 11 | 17.2 |

TABLE 3
Effects of the artificial blood on red blood
cells in mice with hemorrhagic anemia
Hemoglobin (g/dl)

| | 0 h | 20 h | 42 h | 68 h | 92 h |
|---|---|---|---|---|---|
| the artificial | 8.5 | 6.4 | 7.9 | 9.3 | 9.8 |
| blood | | | | | |
| control group I | 8.5 | 5.6 | 6.3 | 7.8 | 9.1 |
| control group II | 8.5 | 5.9 | 6.9 | 8.7 | 9.4 |
| control group III | 8.5 | 5.3 | 6.0 | 7.4 | 8.8 |
| control group IV | 8.5 | 5.3 | 5.8 | 7.6 | 8.8 |
| control group V | 8.4 | 5.2 | 5.9 | 7.8 | 8.6 |
| control group VI | 8.5 | 4.8 | 5.8 | 6.8 | 8.6 |
| control group VII | 8.4 | 4.5 | 4.9 | 5.2 | 8.3 |
| control group | 8.5 | 6.2 | 4.9 | 8.9 | 9.7 |
| VIII | | | | | |
| control group X | 8.4 | 4.3 | 4.8 | 5.2 | 8.2 |

TABLE 4
Comparison of blood oxygen concentration
(SaO₂%) after intravenous infusion

| | Before | the artificial | Perfluorocarbon | physiological |
|---|---|---|---|---|
| | infusion | blood | emulsion | saline |
| 30 min | 84 | 93.3 | 93.4 | 85 |
| 60 min | 84 | 96.6 | 96.8 | 85 |
| 100 min | 85 | 95.3 | 94 | 90.5 |

By intergroup comparison, the experiment shows:

(1) The artificial blood has a certain effect on the recovery of hemorrhagic anemia in control groups I to VII, and the therapeutic effect of the artificial blood is better than that of the control group. This indicates that the synergistic effect of essential amino acids, trace elements, vitamins, nutrients, and growth factors in the artificial blood can stimulate hematopoiesis and expand blood volume.

From the artificial blood and control groups I and VIII, it can be seen that growth factors have a significant promoting effect on increasing hemoglobin content and red blood cell count within 42 hours. This is because human stem cell growth factor SCF and recombinant human erythropoietin EPO can promote the generation of hematopoietic stem cells in the blood, promote red blood cell division, which is more conducive to the transportation of nutrients and other substances, thereby facilitating the recovery of hematopoietic function. Compared to single essential amino acids, trace elements, nutrients, or vitamins, the artificial blood produced by the preparing method of artificial blood has a synergistic effect on improving hematopoietic capacity, and recombinant human platelet growth factor TPO and recombinant human interleukin IL-11 have a certain promoting effect on hemoglobin and blood cell production in the blood. This is because both can not only increase the number of blood platelets, but also promote the expansion of hematopoietic stem cells together with SCF and EPO. For example, IL-11 can help in the GO phase of the hematopoietic stem cell cycle, and TPO can act on progenitor cells and hematopoietic stem cells, thereby promoting the proliferation of red blood cells.

From the artificial blood and control groups I, II, and III, it can be seen that the hemoglobin and red blood cell recovery ability of the artificial blood further decrease after excluding iron, nutrients, and growth factors. Because iron is an important raw material for hemoglobin, the lack of iron will inevitably reduce its synthesis. Hematopoietic stem cells use vitamin C to regulate the abundance of certain chemical modifications on DNA (as part of the epigenome), and they need to consume very high levels of vitamin C. Consuming a large amount of VA can produce yellow acid, which can effectively activate hematopoietic stem cells. Cobalamin in vitamin B12 can promote the development and maturation of red blood cells, promote the microbial synthesis of nucleotides, and keep the hematopoietic function of the body normal. Niacin, total flavonoids, folate, and beta carotene can all promote hematopoietic function to a certain extent and improve anemia.

From control group II and control group IV, it can be seen that besides iron, other trace elements can slightly promote the recovery ability of hemoglobin and red blood cells. This is because other trace elements can also promote hematopoietic function or protect the hematopoietic system, stimulate red blood cell division. From control groups I to VII, it can be seen that although the hematopoietic ability of artificial blood decreases after excluding iron and growth factors, it still slightly improves compared to supplementing only essential amino acids, trace elements, or vitamins. This is only because the synthesis of red blood cells and hemoglobin does not rely solely on a single element, but rather on the comprehensive utilization of various raw materials and the synergistic effect of multiple substances.

(2) In this experiment, due to the blood loss of mice about 40%, there may be a loss of hematopoietic raw material iron. Therefore, the present invention supplements iron agents. From the artificial blood and control group II, it can be seen that reagents containing iron can promote the recovery of red blood cells and hemoglobin within 42 hours. This is because iron is a necessary raw material for the synthesis of hemoglobin and the structural center of hemoglobin. It is a key part responsible for binding with oxygen and plays an important role in the synthesis of red blood cells and oxygen carrying capacity after the artificial blood enters the human body.

On the other hand, iron can promote the synthesis of hemoglobin peptides and increase the amount of hemoglobin. From control groups I, III, IV, V, and VI, it can be seen that amino acids and glucose, as other important raw materials for synthesizing red blood cells, have a promoting and improving effect on the recovery of red blood cells and hemoglobin. And the combination of the two is more effective than using them alone. This is because in addition to the necessary amino acids and iron elements, nutrients play an important role in the energy and promotion of stem cell growth during the process of red blood cell synthesis. For example, Dr. Michhalis Agathocleous from the University of Texas Southwestern Medical Center found that hematopoietic stem cells uptake very high levels of vitamin C, and hematopoietic stem cells use vitamin C to regulate the abundance of certain chemical modifications on DNA (as part of the epigenome).

When hematopoietic stem cells do not take up enough vitamin C, their epigenome can suffer losses in a way that increases their function but also increases the risk of leukemia. For example, research by the German Cancer Research Center and the Heidelberg Institute for Stem Cell Research and Experimental Medicine has found that a deficiency in vitamin A leads to the loss of important hematopoietic stem cells. For example, folate is an important coenzyme for DNA synthesis, folic acid deficiency affects the DNA synthesis of young red blood cells in the bone marrow, leading to a significant slowdown in cell division and proliferation, resulting in developmental disorders of red blood cell nuclei and inhibition of nuclear division. Therefore, the composite preparation of the present invention can better promote the recovery of mice with hemorrhagic anemia.

(3) In the experiment, due to the significant decrease in blood oxygen saturation in mice after blood loss, it was found through the experimental group and comparative examples 1 and 2 that the blood oxygen concentration of the artificial blood and comparative example 1 in this application increased significantly and gradually returned to normal levels at 30 minutes, 60 minutes, and 100 minutes after infusion, while in comparative example 2, the administration of physiological saline only slightly increased the blood oxygen concentration. Moreover, it can be seen from the experimental group and comparative example 1 that the artificial blood of the present invention has a comparable ability to increase blood oxygen, indicating that its oxygen carrying capacity is comparable to that of perfluoroalkane emulsion.

It can be used as a substitute for blood supplementation with higher safety and less residual metabolites.

### III. Clinical Data

Select 100 adult patients diagnosed with iron deficiency anemia, aged between 16 and 65 years old. 100 patients with iron deficiency anemia were divided into two groups based on gender, age, red blood cell count (RBC), and hemoglobin (HB), with 50 patients in each group. They were then randomly assigned to an observation group and a control group.

Inclusion criteria: Patients with iron deficiency anemia whose hemoglobin level is less than 120 g/L.

Exclusion criteria: Individuals under the age of 18 or over 65, pregnant or lactating women, and patients with other organic or psychiatric disorders. Subjects who did not take the test substance according to regulations and have incomplete data may affect the efficacy or safety evaluation.

100 adult patients diagnosed with iron deficiency anemia were randomly divided into experimental group and control group. The experimental group was given continuous oral administration of the artificial blood, while the control group was given a placebo with the same appearance and taste, twice a day, one bottle (45 ml) each time, with an observation period of 30 days. During the trial period, there was no intervention in the lifestyle and diet of the subjects, but other iron containing preparations were avoided.

The experimental results are as follows:

TABLE 5
Comparison of hematopoietic stem cell and hemoglobin
results between experimental group and control
group before and after the experiment

| | Experimental Group | | Control Group | |
|---|---|---|---|---|
| subjects | Before | After | Before | After |
| Hematopoietic Stem Cell | 0.11 | 4.5 | 0.12 | 0.2 |
| (%) | | | | |
| Hemoglobin (g/l) | 88.5 | 108.3 | 89 | 90 |

TABLE 6
Comparison of blood oxygen concentration before and after the
experiment between the experimental group and the control group

| | Experimental Group | | Control Group | |
|---|---|---|---|---|
| subjects | Before | After | Before | After |
| Blood oxygen concentration | 90 | 95 | 90 | 91 |
| (%) | | | | |

The experimental results showed that 50 adult patients diagnosed with iron deficiency anemia in the experimental group showed significant improvement in various blood indicators after continuous use of the artificial blood for 30 days. The amount of hematopoietic stem cells in the blood has significantly improved, with an average increase in hemoglobin content and a slight increase in platelets. The total effective rate of improving nutritional anemia in the experimental group reached 87%. The results indicate that the artificial blood is a combination that promotes cell regeneration, which can stimulate the proportion of hematopoietic stem cells in the blood through oral administration, thereby achieving the function of promoting cell and tissue regeneration. The artificial blood has a significant ability to increase blood oxygen saturation after anemia, and continuous use of the artificial blood can help it recover to normal levels. The Artificial blood has a significant improvement effect on nutritional anemia.

Finally, it should be noted that the above description is only a preferred embodiment of the present invention and is not intended to limit the present invention. Although the present invention has been described in detail with reference to the aforementioned embodiments, those skilled in the art can still modify the technical solutions described in the aforementioned embodiments or equivalently replace some of the technical features. Any modifications, equivalent substitutions, improvements, etc. made within the spirit and principles of the present invention shall be included within the scope of protection of the present invention.

The above content is a further detailed explanation of the present invention based on specific preferred embodiments, and it cannot be assumed that the specific implementation of the present invention is limited to these explanations. For ordinary technical personnel in the technical field to which the present invention belongs, the architecture form can be flexible and varied, and a series of products can be derived without departing from the concept of the present invention. Just making a few simple deductions or substitutions should be considered as falling within the scope of patent protection determined by the submitted claims of the present invention.

What is claimed is:

1. A preparing method for artificial blood, per 100 g of said artificial blood including raw materials as follows: 12-15 g of essential amino acids, 6.5-7.0 g of trace elements, 23-23.5 g of nutrients, and 0.05-0.06 mg of vitamins; comprising following steps:
(1) add said raw materials into a tank reactor, and mix with solvent to obtain a mixture;
(2) add glycerol, lecithin, soybean oil, ethyl cellulose, TritonX-100, and Tween-40 in a high shear milk machine in a mass ratio of 2:2:2:1:0.05:0.05; control said high shear milk machine to react at 20° C. for 30 min, then stir with a microwave for 40 seconds to obtain a white emulsion suspension;
(3) take 100 g of said mixture obtained in step (1), add 1000 ml of physiological saline, and mix well as a diluent; dilute said white emulsion suspension obtained in step (2) with said diluent at a ratio of 1:25, then heat to 90° C. and cool to 10° C. under a vacuum to form an amino acid solution wrapped in emulsion particles;
(4) inject a mixture of medical sterile pure O₂ and O₃ at 2-3 atmospheres of pressure or oxygen undergoing photochemical treatment using a medical high oxygen liquid therapy device at a flow rate of 4-5.5 L/min into said amino acid solution obtained in step (3) for dissolved oxygen treatment for 25 minutes;
(5) measuring said amino acid solution after the dissolved oxygen treatment obtained in step (4) using a blood gas analyzer, and when a partial pressure of oxygen reaches 105 Kpa or more, filling same into an infusion bag at equal pressure to obtain said artificial blood.

2. The preparing method for artificial blood according to claim 1, wherein in 100 g of said artificial blood, said essential amino acids include arginine 0.0058 g lysine 0.0026 g, tyrosine<0.0038 g, leucine 0.0032 g, aspartic acid 0.11 g, valine 0.0036 g, isoleucine 0.0018 g;
said trace elements include vanadium 0.00260 mg, manganese 0.0706 mg, iron 6.60 mg, cobalt 0.00106 mg, nickel less than 0.05 mg, copper less than 0.02 mg, lithium less than 0.02 mg, arsenic 0.00724 mg, selenium less than 0.003 mg, rubidium 0.0434 mg, molybdenum less than 0.003 mg;
said nutrients include total flavone 7.88 mg, folate<0.0015 g, triterpenes<2.61 g on an oleanolic acid basis, fructooligosaccharides<0.103 mg, beta carotene<1.5 μg, fructose 3.4 g, glucose 13.4 g, sucrose 3.7 g, niacin<100 μg;
said vitamins include L (+) ascorbic acid<0.4 mg, vitamin A<0.35 mg, vitamin B12<0.05 mg;
said growth factors include human stem cell growth factor SCF1.5×10-5 mg, recombinant human erythropoietin EPO1.2×10-5 mg, recombinant human platelet growth factor TPO1.2×10-5 mg, recombinant human interleukin IL-11 112.5×10-5 mg.

3. The preparing method for artificial blood according to claim 1, wherein in 100 g of said artificial blood, said essential amino acids include methionine<0.0030 g, glycine<0.0048 g, glutamic acid<0.018 g, leucine<0.0032 g, serine<0.0055 g, valine<0.0036 g, isoleucine<0.0018 g, histidine<0.0018 g;
said trace elements include vanadium 0.00260 mg, manganese 0.0706 mg, iron 6.60 mg, cobalt 0.00106 mg, nickel less than 0.05 mg, copper less than 0.02 mg, lithium less than 0.02 mg, arsenic 0.00724 mg, selenium less than 0.003 mg, rubidium 0.0434 mg, molybdenum less than 0.003 mg;
said nutrients include total flavone 7.88 mg, folate<0.0015 g, triterpenoids<2.61 g, fructooligosaccharides 0.103 mg/100 ml, beta carotene<1.5 μg, fructose 3.4 g, glucose 13.4 g, sucrose 3.7 g, Niacin<100 μg;
said vitamins include L (+) ascorbic acid<0.4 mg, vitamin A<0.35 mg, vitamin B12<0.05 mg;
said growth factors include human stem cell growth factor SCF1.5×10-5 mg, recombinant human erythropoietin EPO1.2×10-5 mg.

4. The preparing method for artificial blood according to claim 1, wherein in 100 g of said artificial blood, said essential amino acids include phenylalanine 0.0044 g, alanine 0.0055 g, proline<0.12 g, leucine 0.0032 g, aspartic acid 0.11 g, valine 0.0036 g, isoleucine 0.0018 g;
said trace elements include vanadium 0.0260 mg, manganese 0.706 mg, iron 66.0 mg, cobalt 0.0106 mg, nickel<0.5 mg, copper<0.2 mg, lithium<0.2 mg, arsenic 0.0724 mg, selenium<0.03 mg, rubidium 0.434 mg, molybdenum<0.03 mg;
said vitamins include L (+) ascorbic acid<0.4 mg, vitamin A<0.35 mg, vitamin B12<0.05 mg;
said growth factors include human stem cell growth factor SCF1.5×10⁻⁵ mg.

5. The preparing method for artificial blood according to claim 1, wherein said trace elements come from organic compounds extracted from food or other forms of compounds that are easily absorbed by a human body.

6. The preparing method for artificial blood according to claim 1, wherein an amount of iron added is 55 mg/100 g.

7. The preparing method for artificial blood according to claim 1, wherein an amount of L (+) ascorbic acid added to the vitamin is 0.35 mg/100 g, and an amount of vitamin A added is 0.30 mg/100 g.

8. The preparing method for artificial blood according to claim 1, wherein in said mixture of pure O₂ and O₃, the ratio of pure O₂ to O₃ is 3.54:1 by volume percentage.
